(19) United States
(12) Patent Application Publication
(10) Pub. No.: US 2024/0206310 A1
(43) Pub. Date: Jun. 20, 2024

(54) COMPOUND AND DEVICE

(71) Applicant: Sumitomo Chemical Co., Ltd., Tokyo (JP)

(72) Inventor: Michal Maciejczyk, Godmanchester (GB)

(73) Assignee: Sumitomo Chemical Co., Ltd., Tokyo (JP)

(21) Appl. No.: 18/518,722

(22) Filed: Nov. 24, 2023

(30) Foreign Application Priority Data
Nov. 28, 2022 (GB) 2217837.0

(51) Int. Cl. H10K 85/10; H10K 30/30; H10K 71/15

(52) U.S. Cl. CPC H10K 85/113; H10K 30/30; H10K 71/15

(57) ABSTRACT

A compound of formula (I) or (II):

A¹-(B¹)x¹-(D¹)y¹-(B¹)x²-A¹ (I)

A¹-(B²)x⁵-(D²)y²-(B³)x³-A²-(B³)x⁴-(D³)y³-(B²)x⁶-A¹ (II)

wherein: A¹ in each occurrence is independently a monovalent electron-accepting group; A² is a divalent electron-accepting group; D¹, D² and D³ independently in each occurrence is an electron-donating group; y¹, y² and y³ are each independently at least 1; B¹, B², and B³ independently in each occurrence is a bridging group; x¹-x⁶ are each independently 0, 1, 2 or 3 with the provisos that: in the case of the compound of formula (I) at least one of x¹ and x² is at least 1 and at least one B¹ is substituted with a fluorinated group; and in the case of the compound of formula (II) at least one of x³, x⁴, x⁵ and x⁶ is at least 1 and at least one occurrence of at least one of B² and B³ is substituted with a fluorinated group. The compound of formula (I) or (II) may be used as an electron-accepting material in a photoresponsive device.

(NO FIGURE)

### RELATED APPLICATIONS

This application claims priority to United Kingdom Patent Application GB 2217837.0, filed Nov. 28, 2022, the contents of which are incorporated herein by reference.

### BACKGROUND

Embodiments of the present disclosure relate to electron-accepting compounds suitable for use in photoresponsive devices, particularly organic photodetectors.

An organic photoresponsive device may contain a photoactive layer of a blend of an electron-donating material and an electron-accepting material between an anode and a cathode. Known electron-accepting materials include fullerenes and non-fullerene acceptors (NFAs).

Examples of NFAs are disclosed in US 2021/0367159, US 2019/157581 and WO2022/129137.

### SUMMARY

The present disclosure provides a compound of formula (I) or (II):

A¹-(B¹)x¹-(D¹)y¹-(B¹)x²-A¹ (I)

A¹-(B²)x⁵-(D²)y²-(B³)x³-A²-(B³)x⁴-(D³)y³-(B²)x⁶-A¹ (II)

wherein:

A¹ in each occurrence is independently a monovalent electron-accepting group;

A² is a divalent electron-accepting group;

D¹, D² and D³ independently in each occurrence is an electron-donating group;

y¹, y² and y³ are each independently at least 1;

B¹, B², and B³ independently in each occurrence is a bridging group;

x¹-x⁶ are each independently 0, 1, 2 or 3 with the provisos that:

in the case of the compound of formula (I) at least one of x¹ and x² is at least 1 and at least one B¹ is substituted with a fluorinated group; and

in the case of the compound of formula (II) at least one of x³, x⁴, x⁵ and x⁶ is at least 1 and at least one occurrence of at least one of B² and B³ is substituted with a fluorinated group.

The present disclosure provides a composition comprising an electron-donating material and an electron-accepting material of formula (I) or (II).

The present disclosure provides an organic electronic device comprising an active layer comprising a compound of formula (I) or (II). Preferably, the organic electronic device is an organic photodetector.

The present disclosure provides a formulation comprising a compound or composition as described herein dissolved or dispersed in one or more solvents.

The present disclosure provides a method of forming an organic electronic device as described herein wherein formation of the active layer comprises deposition of a formulation as described herein onto a surface and evaporation of the one or more solvents.

The present disclosure provides an organic photoresponsive device comprising an anode, a cathode and a photoactive layer disposed between the anode wherein the photoactive layer comprises an electron-donating sub-layer comprising an electron-donating compound and an electron-accepting sub-layer comprising a compound of formula (I′) or (II′) directly adjacent to and in contact with the electron-donating sub-layer:

A¹-(B¹)x¹¹-(D¹)y¹-(B¹)x¹²-A¹ (I′)

A¹-(B²)x¹⁵-(D²)y²-(B³)x¹³-A²-(B³)x¹⁴-(D³)y³-(B²)x¹⁶-A¹ (II′)

wherein:

A¹ in each occurrence is independently a monovalent electron-accepting group;

A² is a divalent electron-accepting group;

D¹, D² and D³ independently in each occurrence is an electron-donating group;

y¹, y² and y³ are each independently at least 1;

B¹, B², and B³ independently in each occurrence is a bridging group;

x¹¹-x¹⁶ are each independently 0, 1, 2 or 3; and the compound of formula (I′) or (II′) is substituted with at least one fluorinated group.

The present disclosure provides method of forming the organic photoresponsive device wherein formation of the electron-accepting sub-layer comprises deposition of a formulation comprising a halogenated solvent and the compound of formula (I′) or (II′) dissolved in the formulation, and wherein formation of the electron-donating sub-layer comprises deposition of a formulation comprising one or more solvents selected from non-halogenated solvents and the electron-donating compound.

### DESCRIPTION OF DRAWINGS

The disclosed technology and accompanying figures describe some implementations of the disclosed technology.

FIG. 1 illustrates an organic photoresponsive device according to some embodiments.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise.” “comprising.” and the like are to be construed in an inclusive sense, as opposed to an exclusive or exhaustive sense; that is to say, in the sense of “including, but not limited to.” Additionally, the words “herein,” “above,” “below.” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers may be present. References to a layer “on” another layer when used in this application means that the layers are in direct contact. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present disclosure provides compounds comprising electron-accepting compounds of formula (I), (II), (I′) or (II′).

Each of the electron-accepting groups A¹ and A² has a lowest unoccupied molecular orbital (LUMO) level that is deeper (i.e., further from vacuum) than the LUMO of any of the electron-donating groups D¹, D² or D³, preferably at least 1 eV deeper. The LUMO levels of electron-accepting groups and electron-donating groups may be as determined by modelling the LUMO level of these groups, in which each bond to adjacent group is replaced with a bond to a hydrogen atom. Modelling may be performed using Gaussian09 software available from Gaussian using Gaussian09 with B3LYP (functional) and LACVP* (Basis set).

The compound of formula (I) or (II) has a bridging group B¹, B² or B³ which is substituted with one or more fluorinated groups.

In the case of compounds of formula (I), at least one of x¹ and x² is at least 1 and at least one B¹ is substituted with at least one fluorinated group. In some embodiments, A¹ and/or D¹ is also substituted with at least one fluorinated group.

In the case of compounds of formula (II), at least one of x³-x⁶ is at least 1 and at least one of B² and B³ is substituted with at least one fluorinated group. More preferably, at least one of x⁵ and x⁶ is at least one and at least one B² is substituted with at least one fluorinated group. In some embodiments, A¹, A², D² and/or D³ is also substituted with at least one fluorinated group.

The compound of formula (I′) or (II′) is substituted with at least one fluorinated group. Preferably, at least one of the donor groups D¹, D² and D³ and/or a bridging group B¹, B² or B³ is substituted with at least one fluorinated group.

In the case of compounds of formula (I′), D¹ is substituted with at least one fluorinated group; B¹ may or may not be present; and B¹ (if present) and/or A¹ may or may not be substituted with at least one fluorinated group.

In the case of compounds of formula (II′), D² and/or D³ is substituted with at least one fluorinated group; B² and B³ may or may not be present; and B² and B³ (if present), A¹, and/or A² may or may not be substituted with at least one fluorinated group.

A fluorinated group as described herein may enhance solubility of a compound of formula (I) (II), (I′) or (II′) in a halogenated solvent, preferably a fluorinated solvent, as compared to a corresponding compound of formula (I) (II), (I′) or (II′) in which fluorinated groups are absent.

Moreover, the number, identity and position of fluorinated groups as described herein may be selected so as to tune the electronic properties of a compound of formula (I) (II), (I′) or (II′).

### Bridging Units

Bridging units B¹, B² and B³ of formulae (I) (II), (I′) and (II′) are preferably each selected from vinylene, arylene, heteroarylene, arylenevinylene and heteroarylenevinylene wherein the arylene and heteroarylene groups are monocyclic or bicyclic groups, each of which may be unsubstituted or substituted with one or more substituents. Preferably, at least one B¹ in the case of formula (I) or (I′) or at least one occurrence of at least one of B² and B³ in the case of formula (II) or (II′) is substituted with a fluorinated group as described herein.

A “fluorinated group” as described herein is an organic residue including one or more fluorine atoms bound to a carbon atom. Preferably, the fluorinated group is a perfluorinated group.

Preferably, each bridging unit of formula (I) or (I′) or each bridging unit of formula (II) or (II′) is substituted with at least one fluorinated group, preferably one or two fluorinated groups.

Preferred fluorinated groups are:
partially fluorinated or perfluorinated C₁₋₂₀ alkyl in which one or more non-adjacent C atoms of a C₂₋₂₀ alkyl may be replaced with O, S, NR⁶, Si(R⁴)₂, CO, COO or CONR⁶ wherein R⁶ is H or a substituent and each R⁴ is independently a substituent, and
phenyl which is substituted with at least one F or partially fluorinated C₁₋₂₀ alkyl in which one or more non-adjacent C atoms of a C₂₋₂₀ alkyl may be replaced with O, S, NR⁶, Si(R⁴)₂, CO, COO or CONR⁶.

Optionally, each R⁶ of any NR⁶, CONR⁶ or PR⁶ described anywhere herein is independently selected from H; C₁₋₂₀ alkyl wherein one or more non-adjacent C atoms other than the C atom bound to N or P may be replaced with O, S, NR¹¹, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms of the alkyl may be replaced with O, S, NR¹¹, COO or CO and one or more H atoms of the alkyl may be replaced with F wherein R¹¹ is H or a C₁₋₂₀ hydrocarbyl group.

Each R⁴ is preferably a C₁₋₂₀ hydrocarbyl group.

A C₁₋₂₀ hydrocarbyl group as described anywhere is preferably selected from C₁₋₂₀ alkyl; unsubstituted phenyl; and phenyl substituted with one or more C₁₋₁₂ alkyl groups

By “partially fluorinated” alkyl as used herein is meant alkyl substituted with at least one F atom.

By “partially fluorinated” phenyl as used herein is meant phenyl substituted with at least one group selected from F and an at least partially fluorinated C₁₋₂₀ alkyl.

Preferably, in a fluorinated C₁₋₂₀ alkyl group as described herein at least 50% of the H atoms of the C₁₋₂₀ alkyl, and optionally all of the H atoms, are replaced with F.

Optionally, B¹, B² and B³ are, independently in each occurrence, selected from units of formulae (VIa)-(VIo):

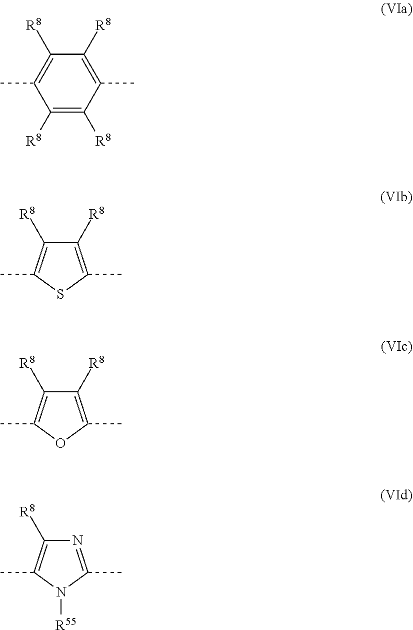

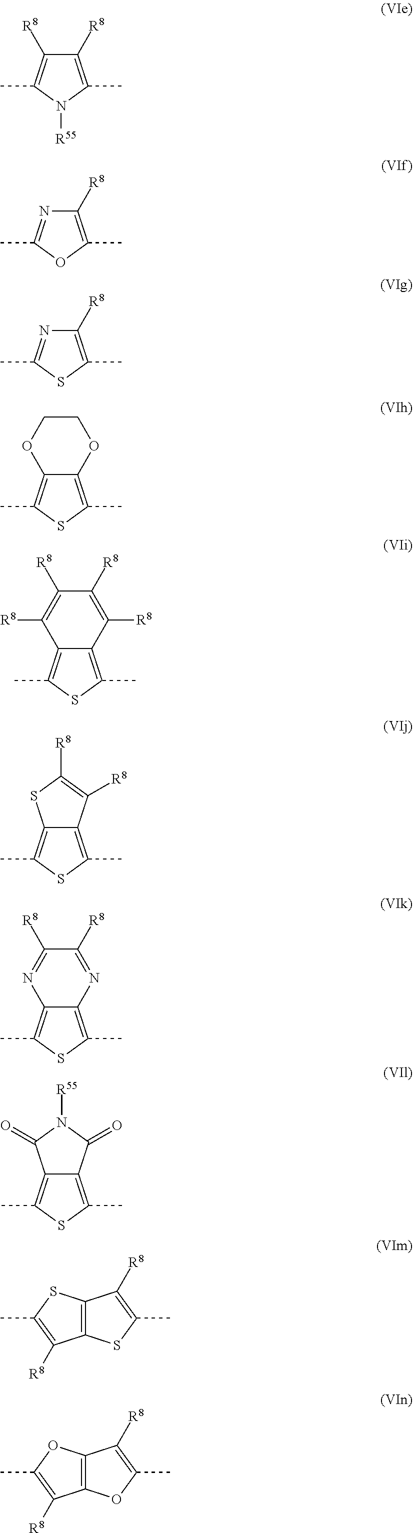

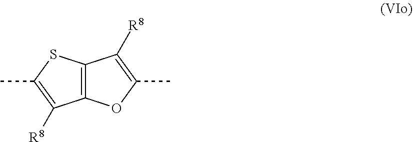

wherein R⁵⁵ is H or a substituent, optionally H or a C₁₋₂₀ hydrocarbyl group; and R⁸ in each occurrence is independently H or a substituent.

Preferably, at least one of B¹, B² and B³ selected from formulae (VIa)-(VIo) has at least one fluorinated group R⁸ wherein the fluorinated group is as described herein.

In some embodiments, each R⁸ is selected from H and a fluorinated group as described herein.

In some embodiments, each R⁸ is selected from H; a fluorinated group; and a substituent selected from F; CN; NO₂; C₁₋₂₀ alkyl wherein one or more non-adjacent C atoms may be replaced with O, S, NR⁶, COO or CO; phenyl which is unsubstituted or substituted with one or more substituents; and —B(R¹⁴)₂ wherein R¹⁴ in each occurrence is a substituent, optionally a C₁₋₂₀ hydrocarbyl group. Substituents of a phenyl group R⁸ may be selected from C₁₋₁₂ alkyl and C₁₋₁₂ alkoxy.

If present, non-fluorinated groups R⁸ are preferably selected from C₁₋₂₀ alkyl or C₁₋₁₉ alkoxy.

R⁸ groups of formulae (VIa), (VIb) and (VIc) may be linked to form an optionally substituted bicyclic ring. Optionally, the bicyclic ring is substituted with one or more fluorinated groups.

In compounds of formula (I), each x¹ is preferably 1.

In compounds of formula (I′), each x¹ is preferably 0 or 1.

In compounds of formula (II), x³ and x⁴ are each preferably 0 and x⁵ and x⁶ are each preferably 1.

In compounds of formula (II′), x³ and x⁴ are each preferably 0 and x⁵ and x⁶ are each preferably 0 or 1.

### Electron-Accepting Groups A¹

The monovalent acceptor groups A¹ may each independently be selected from any such units known to the skilled person. A¹ may be the same or different, preferably the same. Exemplary monovalent acceptor groups include, without limitation, groups of formulae (IXa)-(IXq)

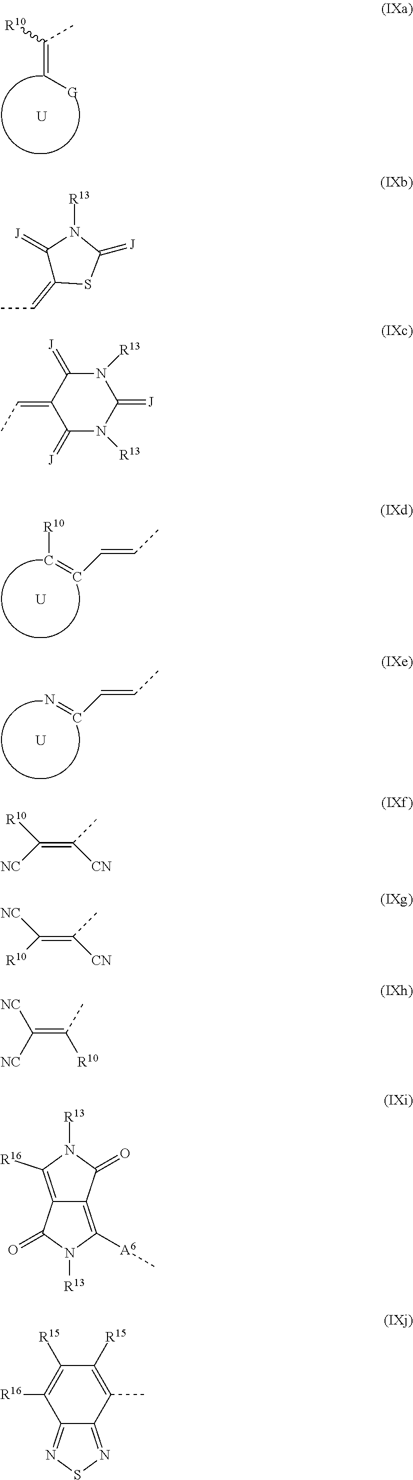

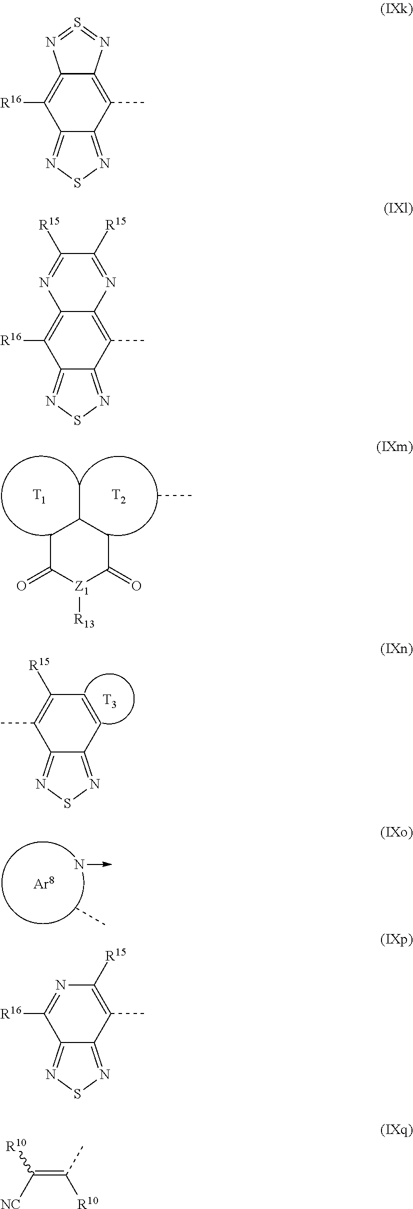

U is a 5- or 6-membered ring which is unsubstituted or substituted with one or more substituents and which may be fused to one or more further rings.

G is C═O, C═S SO, SO₂, NR³³ or C(R³³)₂ wherein R³³ is CN or COOR⁴⁰. G is preferably C═O or SO₂, more preferably C═O.

The N atom of formula (IXe) may be unsubstituted or substituted.

R¹⁰ is H or a substituent, preferably a substituent selected from the group consisting of C₁₋₁₂ alkyl wherein one or more non-adjacent C atoms may be replaced with O, S, NR⁶, COO or CO and one or more H atoms of the alkyl may be replaced with F; and an aromatic group, optionally phenyl, which is unsubstituted or substituted with one or more substituents selected from F and C₁₋₁₂ alkyl wherein one or more non-adjacent C atoms may be replaced with O, S, NR₆, COO or CO.

Preferably, R¹⁰ is H.

J is O or S, preferably O.

R¹³ in each occurrence is a substituent, optionally C₁₋₁₂ alkyl wherein one or more non-adjacent C atoms may be replaced with O, S, NR⁶, COO or CO and one or more H atoms of the alkyl may be replaced with F.

R¹⁵ in each occurrence is independently H; F; C₁₋₁₂ alkyl wherein one or more non-adjacent C atoms may be replaced with O, S, NR⁶, COO or CO and one or more H atoms of the alkyl may be replaced with F; aromatic group Ar², optionally phenyl, which is unsubstituted or substituted with one or more substituents selected from F and C₁₋₁₂ alkyl wherein one or more non-adjacent C atoms may be replaced with O, S, NR⁶, COO or CO; or a group selected from:

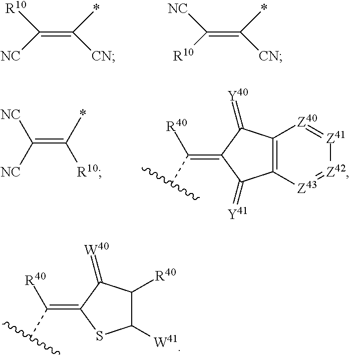

R¹⁶ is H or a substituent, preferably a substituent selected from:
(Ar³)_{w} wherein Ar³ in each occurrence is independently an unsubstituted or substituted aryl or heteroaryl group, preferably thiophene, and w is 1, 2 or 3;

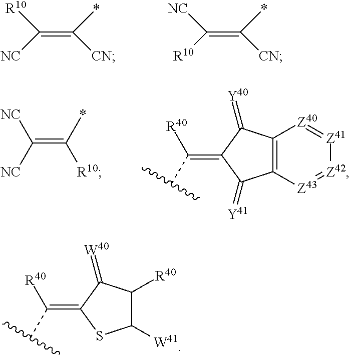

and

C₁₋₁₂ alkyl wherein one or more non-adjacent C atoms may be replaced with O, S, NR⁶, COO or CO and one or more H atoms of the alkyl may be replaced with F.

Ar⁶ is a 5-membered heteroaromatic group, preferably thiophene or furan, which is unsubstituted or substituted with one or more substituents.

Substituents of Ar³ and Ar⁶, where present, are optionally selected from C₁₋₁₂ alkyl wherein one or more non-adjacent C atoms may be replaced with O, S, NR⁶, COO or CO and one or more H atoms of the alkyl may be replaced with F.

T¹, T² and T³ each independently represent an aryl or a heteroaryl ring, optionally benzene, which may be fused to one or more further rings. Substituents of T¹, T² and T³, where present, are optionally selected from non-H groups of R²⁵. In a preferred embodiment, T³ is benzothiadiazole.

Z¹ is N or P.

Ar⁸ is a fused heteroaromatic group which is unsubstituted or substituted with one or more substituents, optionally one or more non-H substituents R¹⁰, and which is bound to an aromatic C atom of B¹ or B² and to a boron substituent of B¹ or B².

Preferred groups A¹ are groups having a non-aromatic carbon-carbon bond which is bound directly to D¹ of formula (I) or D² or D³ of formula (II) or, if present to B¹ of formula (I) or B² of formula (II).

Preferably at least one A¹, preferably both groups A¹, are a group of formula (IXa-1):

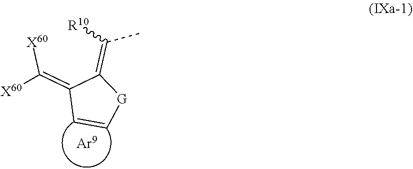

wherein:

G is as described above and is preferably C═O or SO₂, more preferably C═O;

R¹⁰ is as described above;

Ar⁹ is an unsubstituted or substituted monocyclic or fused aromatic or heteroaromatic group, preferably benzene or a monocyclic or bicyclic heteroaromatic group having C or N ring atoms only; and

X⁶⁰ are each independently CN, CF³ or COOR⁴⁰ wherein R⁴⁰ in each occurrence is H or a substituent, preferably H or a C₁₋₂₀ hydrocarbyl group. Preferably, each X⁶⁰ is CN.

Ar⁹ may be unsubstituted or substituted with one or more substituents. Substituents of Ar⁹ are preferably selected from groups R¹² as described below.

Optionally, the group of formula (IXa-1) has formula (IXa-2):

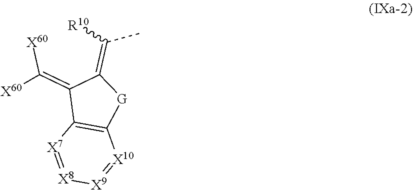

each X⁷-X¹⁰ is independently CR¹² or N wherein R¹² in each occurrence is H or a substituent selected from C₁₋₂₀ hydrocarbyl and an electron withdrawing group. Preferably, the electron withdrawing group is F, Cl, Br or CN, more preferably F, Cl or CN; and for example For CN.

The C₁₋₂₀ hydrocarbyl group R¹² may be selected from C₁₋₂₀ alkyl; unsubstituted phenyl; and phenyl substituted with one or more C₁₋₁₂ alkyl groups.

In a particularly preferred embodiment, each of X⁷-X¹⁰ is CR¹² and each R¹² is independently selected from H or an electron-withdrawing group, preferably H, F or CN. According to his embodiment, R¹² of X⁸ and X⁹ is an electron-withdrawing group, preferably F or CN.

Exemplary groups of formula (IXd) include:

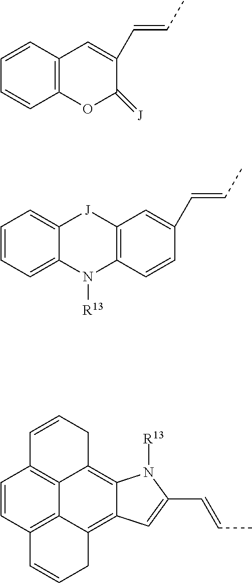

Exemplary groups of formula (IXe) include:

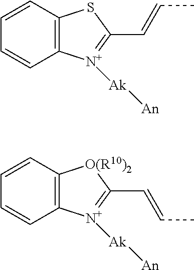

An exemplary group of formula (IXq) is:

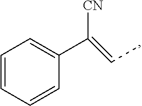

An exemplary group of formula (IXg) is:

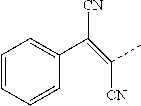

An exemplary group of formula (IXj) is:

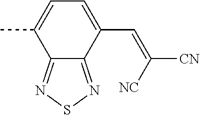

wherein Ak is a C₁₋₁₂ alkylene chain in which one or more C atoms may be replaced with O, S, NR⁶, CO or COO; An is an anion, optionally —SO₃; and each benzene ring is independently unsubstituted or substituted with one or more substituents selected from substituents described with reference to R^{10.}

Exemplary groups of formula (IXm) are:

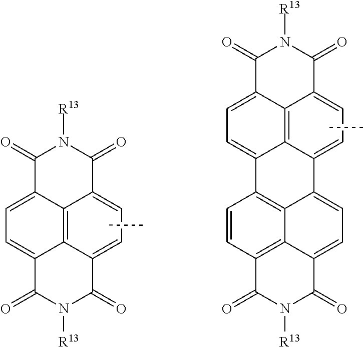

An exemplary group of formula (IXn) is:

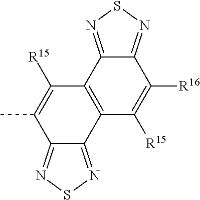

Groups of formula (IXo) are bound directly to a bridging group B¹ or B² substituted with a group of formula —B(R¹⁴)₂ wherein R¹⁴ in each occurrence is a substituent, optionally a C₁₋₂₀ hydrocarbyl group; →is a bond to the boron atom —B(R¹⁴)₂; and — is a C—C bond between formula (IXo) and the bridging group.

Optionally, R¹⁴ is selected from C₁₋₁₂ alkyl; unsubstituted phenyl; and phenyl substituted with one or more C₁₋₁₂ alkyl groups.

The group of formula (IXo), the B¹ or B² group and the B(R¹⁴)₂ substituent of B¹ or B² may be linked together to form a 5- or 6-membered ring.

Ontionally groups of formula (IXo) are selected from:

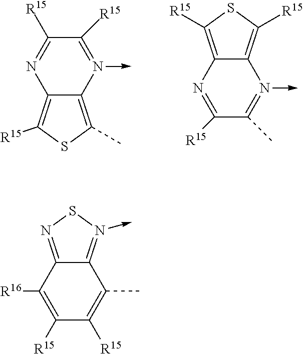

### Acceptor Unit A²

A² is preferably a fused heteroaromatic group comprising at least 2 fused rings, preferably at least 3 fused rings.

In some embodiments, A² of formula (II) is a group of formula (VIII):

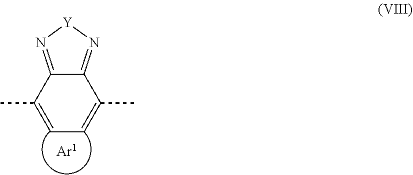

wherein:

Ar¹ is an aromatic or heteroaromatic group; and

Y is O, S, NR⁶ or R⁷—C═C—R⁷ wherein R⁷ in each occurrence is independently H or a substituent wherein two substituents R⁷ may be linked to form a monocyclic or polycyclic ring; and R⁶ is H or a substituent.

In the case where A² is a group of formula (VIII), Ar¹ may be a monocyclic or polycyclic heteroaromatic group which is unsubstituted or substituted with one or more R⁹ groups wherein

R⁹ in each occurrence is independently a substituent.

Preferred R⁹ groups are selected from

F;

CN;

NO₂;

C₁₋₂₀ alkyl wherein one or more non-adjacent C atoms may be replaced with O, S, NR¹⁷ wherein R¹⁷ is a C₁₋₁₂ hydrocarbyl, COO or CO and one or more H atoms of the alkyl may be replaced with F;

an aromatic or heteroaromatic group, preferably phenyl, which is unsubstituted or substituted with one or more substituents; and

a group selected from

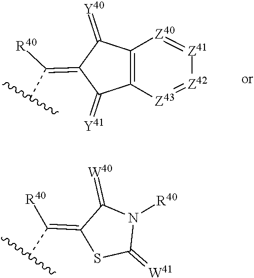

wherein Z⁴⁰, Z⁴¹, Z⁴² and Z⁴³ are each independently CR¹³ or N wherein R¹³ in each occurrence is Hor a substituent, preferably a C₁₋₂₀ hydrocarbyl group; Y⁴⁰ and Y⁴¹ are each independently O, S, NX⁷¹ wherein X⁷¹ is CN or COOR⁴⁰; or CX⁶⁰X⁶¹ wherein X⁶⁰ and X⁶¹ is independently CN, CF³ or COOR⁴⁰; W⁴⁰ and W⁴¹ are each independently O, S, NX⁷¹ or CX⁶⁰X⁶¹ wherein X⁶⁰ and X⁶¹ is independently CN, CF₃ or COOR⁴⁰; and R⁴⁰ in each occurrence is H or a substituent, preferably H or a C₁₋₂₀ hydrocarbyl group. Exemplary substituents of an aromatic or heteroaromatic group R⁹ are F, CN, NO₂, and C₁₋₁₂ alkyl wherein one or more non-adjacent C atoms may be replaced with O, S, NR⁶, COO or CO and one or more H atoms of the alkyl may be replaced with F.

R¹⁷ as described anywhere herein may be, for example, C₁₋₁₂ alkyl, unsubstituted phenyl; or phenyl substituted with one or more C₁₋₆ alkyl groups.

If a C atom of an alkyl group as described anywhere herein is replaced with another atom or group, the replaced C atom may be a terminal C atom of the alkyl group or a non-terminal C-atom.

By “non-terminal C atom” of an alkyl group as used anywhere herein means a C atom other than the C atom of the methyl group at the end of an n-alkyl chain or the C atoms of the methyl groups at the ends of a branched alkyl chain.

If a terminal C atom of a group as described anywhere herein is replaced then the resulting group may be an anionic group comprising a countercation, e.g., an ammonium or metal countercation, preferably an ammonium or alkali metal cation.

A C atom of an alkyl substituent group which is replaced with another atom or group as described anywhere herein is preferably a non-terminal C atom, and the resultant substituent group is preferably non-ionic.

Exemplary monocyclic heteroaromatic groups Ar¹ are oxadiazole, thiadiazole, triazole and 1,4-diazine which is unsubstituted or substituted with one or more substituents. Thiadiazole is particularly preferred.

Exemplary polycyclic heteroaromatic groups Ar¹ are groups of formula (V):

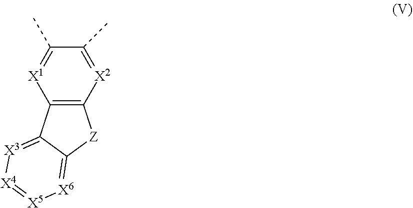

X¹ and X², are each independently selected from N and CR¹⁰ wherein R¹⁰ is H or a substituent, optionally H or a substituent R⁹ as described above.

X³, X⁴, X⁵ and X⁶ are each independently selected from N and CR¹⁰ with the proviso that at least one of X³, X⁴, X⁵ and X⁶ is CR¹⁰.

Z is selected from O, S, SO₂, NR⁶, PR⁶, C(R¹⁰)₂, Si(R¹⁰)₂ C═O, C═S and C═C(R⁵)₂ wherein R¹⁰ is as described above; R⁶ is H or a substituent; and R⁵ in each occurrence is an electron-withdrawing group.

Preferably, each R⁵ is CN, COOR⁴⁰; or CX⁶⁰X⁶¹ wherein X⁶⁰ and X⁶¹ is independently CN, CF₃ or COOR⁴⁰ and R⁴⁰ in each occurrence is H or a substituent, preferably H or a C₁₋₂₀ hydrocarbyl group.

A² groups of formula (VIII) are preferably selected from groups of formulae (VIIIa) and (VIIIb):

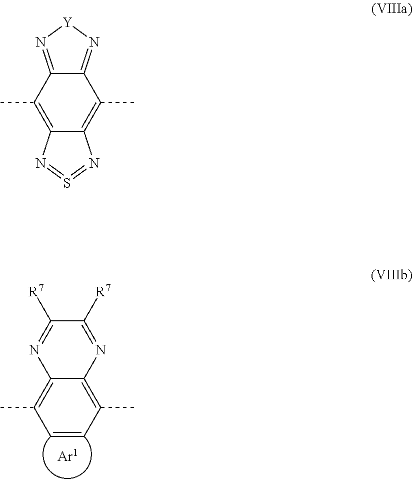

For compounds of formula (VIIIb), the two R⁷ groups may or may not be linked.

Preferably, when the two R⁷ groups are not linked each R⁷ is independently selected from H; F; CN; NO₂; C₁₋₂₀ alkyl wherein one or more non-adjacent C atoms may be replaced with O, S, NR⁶, CO, COO, NR⁶, PR⁶, or Si(R¹⁰)₂ wherein R¹⁰ and R⁶ are as described above and one or more H atoms may be replaced with F; and aryl or heteroaryl, preferably phenyl, which may be unsubstituted or substituted with one or more substituents. Substituents of the aryl or heteroaryl group may be selected from one or more of F; CN; NO₂; and C₁₋₂₀ alkyl wherein one or more non-adjacent C atoms may be replaced with O, S, NR⁶, CO, COO and one or more H atoms may be replaced with F.

Preferably, when the two R⁷ groups are linked, the group of formula (VIIIb) has formula (VIIIb-1) or (VIIIb-2):

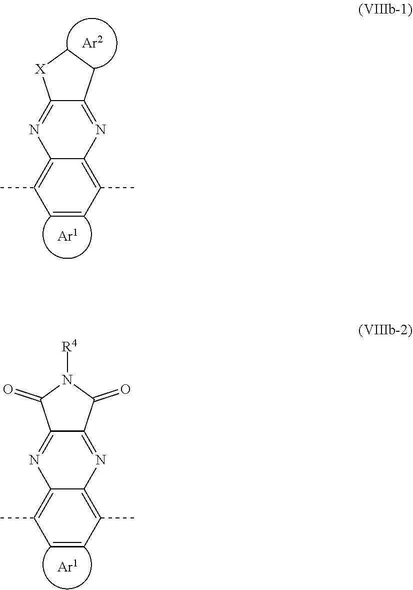

Ar² is an aromatic or heteroaromatic group, preferably benzene, which is unsubstituted or substituted with one or more substituents. Ar² may be unsubstituted or substituted with one or more substituents R² as described above.

X is selected from O, S, SO₂, NR⁶, PR⁶, C(R¹⁰)₂, Si(R¹⁰)₂C—O, C═S and C—C(R⁵)₂ wherein R¹⁰, R⁶ and R⁵ are as described above.

Exemplary electron-accepting groups of formula (VIII) include, without limitation:

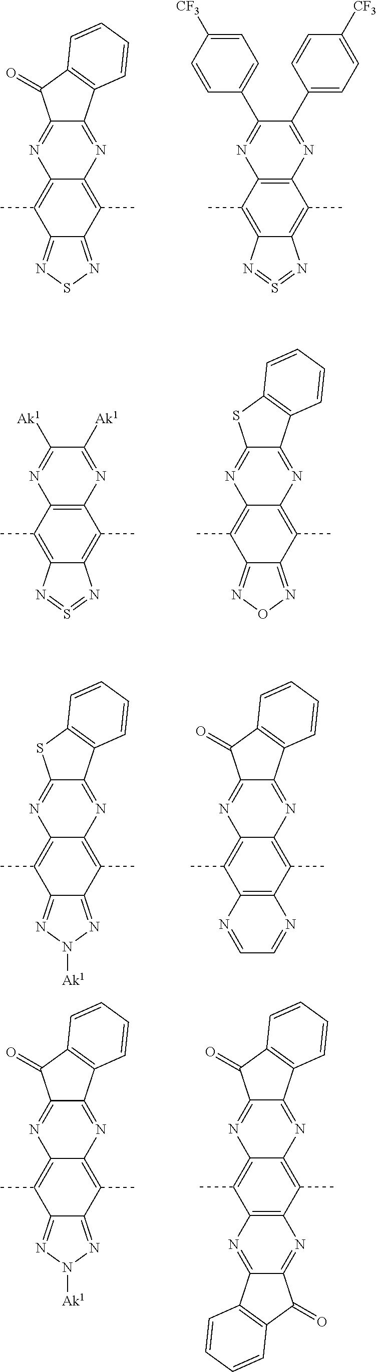

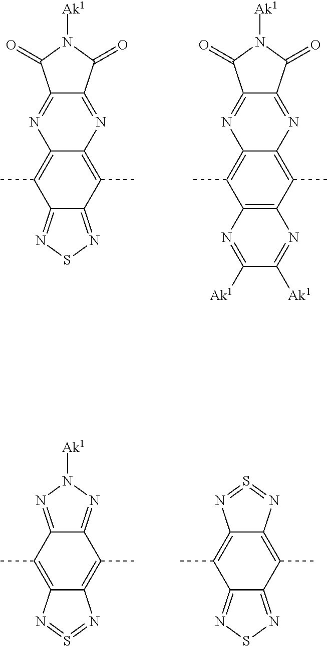

wherein Ak¹ is a C₁₋₂₀ alkyl group

Divalent electron-accepting groups A² other than formula (VIII) are optionally selected from formulac (IVa)-(IVj)

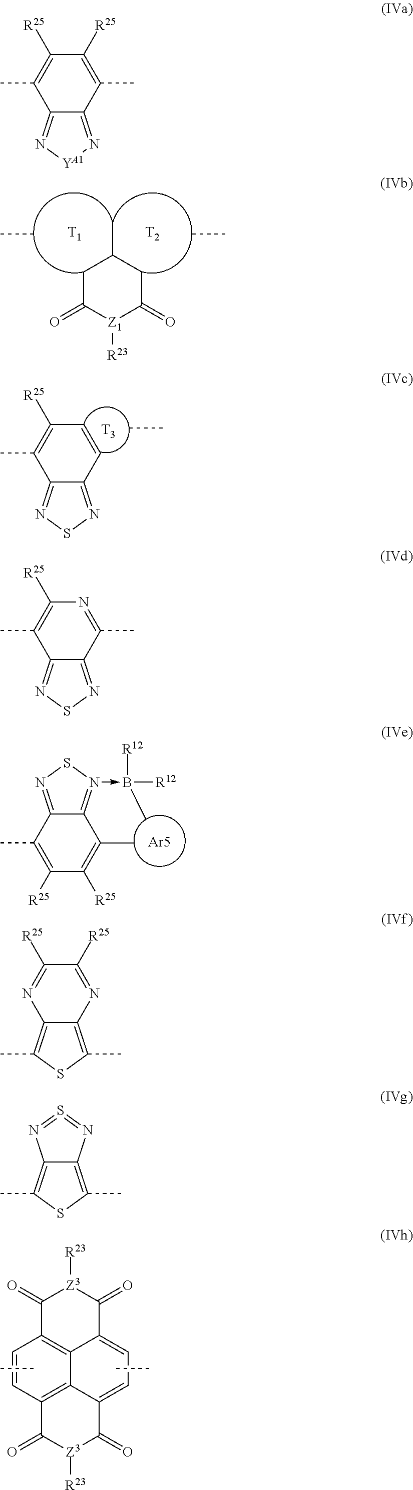

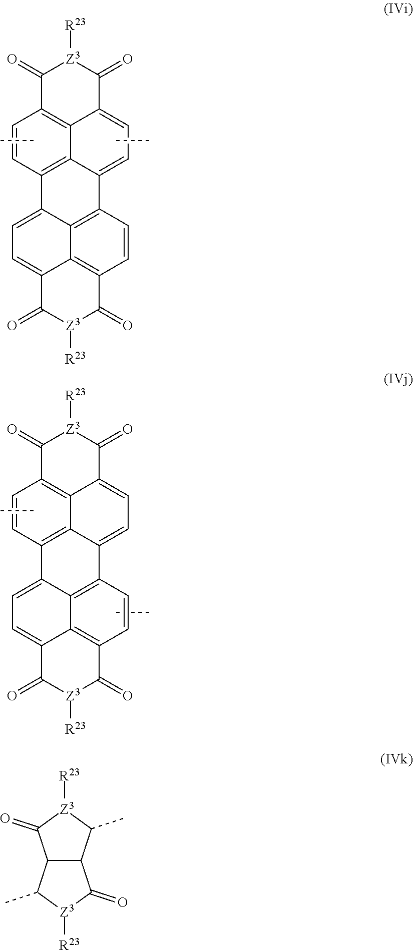

Y^{A1} is O or S, preferably S.

R²³ in each occurrence is a substituent, optionally C₁₋₁₂ alkyl wherein one or more non-adjacent C atoms other than the C atom attached to Z³ may be replaced with O, S, NR⁶, COO or CO and one or more H atoms of the alkyl may be replaced with F.

R²⁵ in each occurrence is independently H; F; CN; NO²; C₁₋₁₂ alkyl wherein one or more non-adjacent C atoms may be replaced with O, S, NR⁶, COO or CO and one or more H atoms of the alkyl may be replaced with F; an aromatic group, optionally phenyl, which is unsubstituted or substituted with one or more substituents selected from F and C₁₋₁₂ alkyl wherein one or more non-adjacent C atoms may be replaced with O, S, NR⁶, COO or CO; or

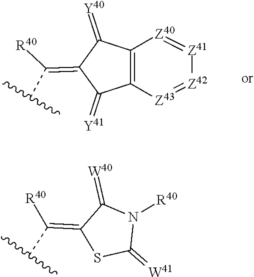

wherein Z⁴⁰, Z⁴¹, Z⁴² and Z⁴³ are each independently CR¹³ or N wherein R¹³ in each occurrence is H or a substituent, preferably a C₁₋₂₀ hydrocarbyl group;

Y⁴⁰ and Y⁴¹ are each independently O, S, NX⁷¹ wherein X⁷¹ is CN or COOR⁴⁰; or CX⁶⁰X⁶¹ wherein X⁶⁰ and X⁶¹ is independently CN, CF₃ or COOR⁴⁰;

W⁴⁰ and W⁴¹ are each independently O, S, NX⁷¹ wherein X⁷¹ is CN or COOR⁴⁰; or CX⁶⁰X⁶¹ wherein X⁶⁰ and X⁶¹ is independently CN, CF₃ or COOR⁴⁰; and R⁴⁰ in each occurrence is H or a substituent, preferably H or a C₁₋₂₀ hydrocarbyl group. Z³ is N or P. T¹, T² and T³ each independently represent an aryl or a heteroaryl ring, optionally benzene, which may be fused to one or more further rings. Substituents of T¹, T² and T³, where present, are optionally selected from non-H groups of R²⁵. In a preferred embodiment, T³ is benzothiadiazole.

R¹² in each occurrence is a substituent, preferably a C₁₋₂₀ hydrocarbyl group.

Ar⁵ is an arylene or heteroarylene group, optionally thiophene, fluorene or phenylene, which may be unsubstituted or substituted with one or more substituents, optionally one or more non-H groups selected from R²⁵.

Electron-Donating Groups D¹, D² and D³

Electron-donating groups preferably are fused aromatic or heteroaromatic groups, more preferably fused heteroaromatic groups containing three or more rings. Particularly preferred electron-donating groups comprise fused thiophene or furan rings, optionally fused rings containing thiophene or furan rings and one or more rings selected from benzene, cyclopentadiene, tetrahydropyran, tetrahydrothiopyran and piperidine rings, each of said rings being unsubstituted or substituted with one or more substituents.

Exemplary electron-donating groups D¹, D² and D³ include groups of formulae (VIIa)-(VIIm):

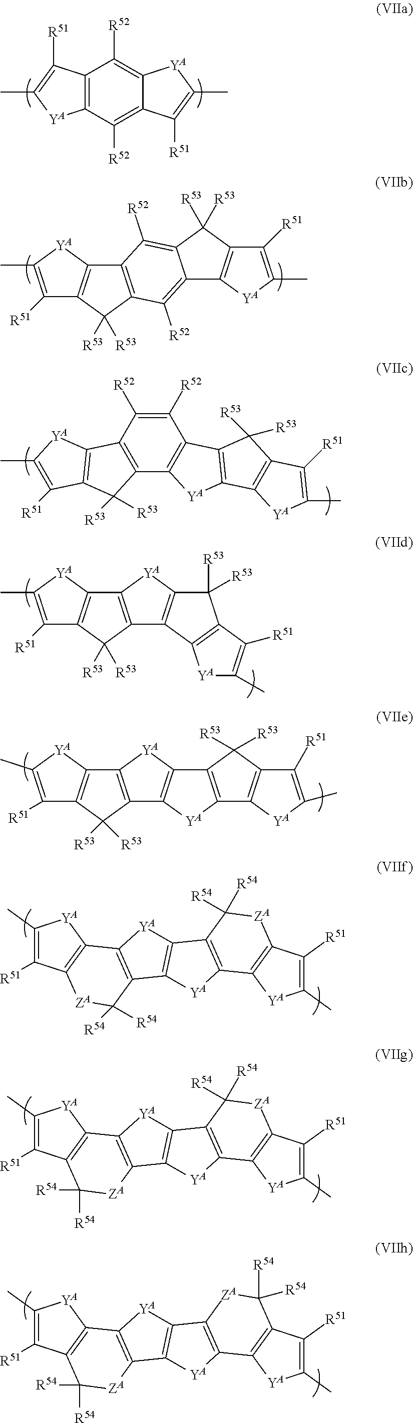

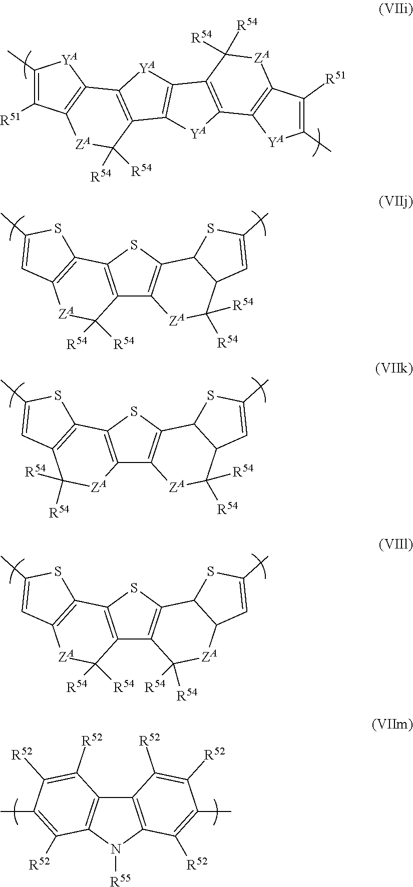

wherein Y^{A} in each occurrence is independently O, S or NR⁵⁵; Z^{A} in each occurrence is O, CO, S, NR⁵⁵ or C(R⁵⁴)₂; R⁵¹, R⁵² R⁵⁴ and R⁵⁵ independently in each occurrence is H or a substituent; R⁵³ independently in each occurrence is a substituent; and Ar⁴ is an optionally substituted monocyclic or fused heteroaromatic group.

Optionally, R⁵¹ and R⁵² independently in each occurrence are selected from H; F; C₁₋₂₀ alkyl wherein one or more non-adjacent C atoms may be replaced with O, S, NR⁶, COO or CO and one or more H atoms of the alkyl may be replaced with F; and an aromatic or heteroaromatic group Ar³ which is unsubstituted or substituted with one or more substituents.

In some embodiments, Ar³ may be an aromatic group, e.g., phenyl.

Ar⁴ is preferably selected from optionally substituted oxadiazole, thiadiazole, triazole, and 1,4-diazine. In the case where Ar⁴ is 1,4-diazine, the 1,4-diazine may be fused to a further heterocyclic group, optionally a group selected from optionally substituted oxadiazole, thiadiazole, triazole, 1,4-diazine and succinimide.

The one or more substituents of Ar³, if present, may be selected from C₁₋₁₂ alkyl wherein one or more non-adjacent C atoms may be replaced with O, S, NR⁶, COO or CO and one or more H atoms of the alkyl may be replaced with F.

Preferably, each R⁵⁴ is selected from the group consisting of:

H;

F;

linear, branched or cyclic C₁₋₂₀ alkyl wherein one or more non-adjacent C atoms may be replaced by O, S, NR¹⁷, CO or COO wherein R¹⁷ is a C₁₋₁₂ hydrocarbyl and one or more H atoms of the C₁₋₂₀ alkyl may be replaced with F; and

a group of formula (Ak)u-(Ar⁷)v wherein Ak is a C₁₋₂₀ alkylene chain in which one or more non-adjacent C atoms may be replaced with O, S, NR⁶, CO or COO; u is 0 or 1; Ar⁷ in each occurrence is independently an aromatic or heteroaromatic group which is unsubstituted or substituted with one or more substituents; and v is at least 1, optionally 1, 2 or 3.

Substituents of Ar^{7,} if present, are preferably selected from F; Cl; NO₂; CN; and C₁₋₂₀ alkyl wherein one or more non-adjacent C atoms may be replaced with O, S, NR⁶, CO or COO and one or more H atoms may be replaced with F. Preferably, Ar⁷ is phenyl.

Preferably, each R⁵¹ is H.

Optionally, R⁵³ independently in each occurrence is selected from C₁₋₂₀ alkyl wherein one or more non-adjacent C atoms may be replaced with O, S, NR⁶, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms may be replaced with O, S, NR⁶, COO or CO and one or more H atoms of the alkyl may be replaced with F.

Preferably, R⁵⁵ as described anywhere herein is H or C₁₋₃₀ hydrocarbyl group.

In a preferred embodiment, D¹ of the compound of formula (I) is a group of formula (VIIe).

In some embodiments, y¹ of formula (I) is 1.

In some embodiments, y² and y³ of formula (II) are each 1.

In some embodiments, y¹ of formula (I) or at least one of y² and y³ of formula (II) is greater than 1. In these embodiments, the chain of D¹, D² or D³ groups, respectively, may be linked in any orientation.

Exemplary compounds of formula (I) include, without limitation:

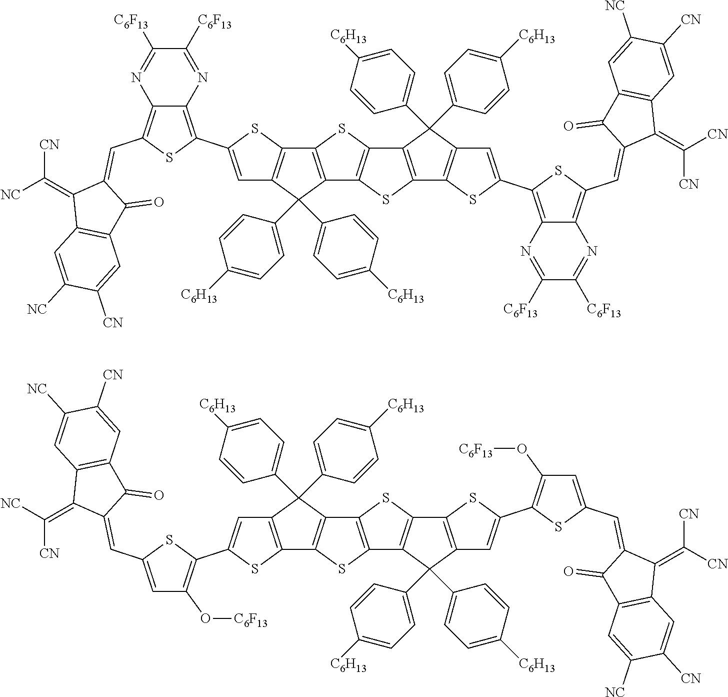

Exemplary compounds of formula (I′) include, without limitation:

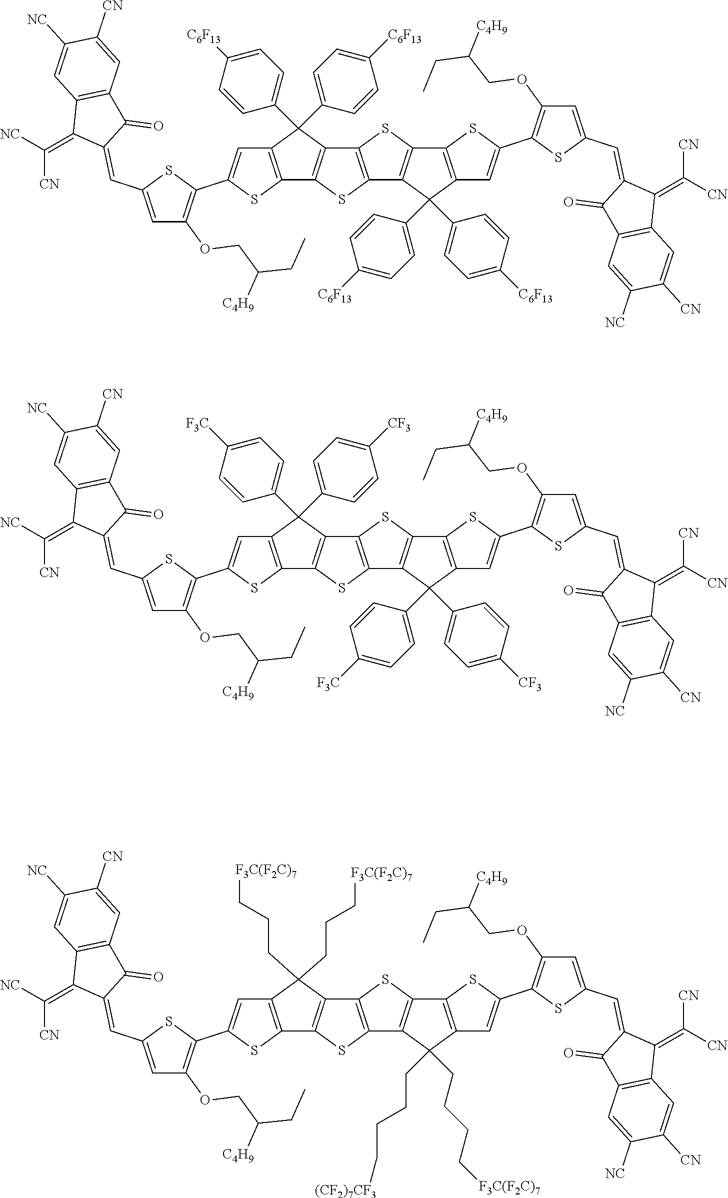

### Electron-Donating Material

Exemplary electron-donating materials suitable for a bulk heterojunction layer or an electron-donating sub-layer are disclosed in, for example, WO2013/051676, the contents of which are incorporated herein by reference.

The electron-donating material may be a non-polymeric or polymeric material.

In a preferred embodiment the electron-donating material is an organic conjugated polymer, which can be a homopolymer or copolymer including alternating, random or block copolymers. The conjugated polymer is preferably a donor-acceptor polymer comprising alternating electron-donating repeat units and electron-accepting repeat units.

Preferred are non-crystalline or semi-crystalline conjugated organic polymers.

Further preferably the electron-donating polymer is a conjugated organic polymer with a low bandgap, typically between 2.5 eV and 1.5 eV, preferably between 2.3 eV and 1.8 eV.

Optionally, the electron-donating polymer has a HOMO level no more than 5.5 eV from vacuum level. Optionally, the electron-donating polymer has a HOMO level at least 4.1 eV from vacuum level. As exemplary electron-donating polymers, polymers selected from conjugated hydrocarbon or heterocyclic polymers including polyacene, polyaniline, polyazulene, polybenzofuran, polyfluorene, polyfuran, polyindenofluorene, polyindole, polyphenylene, polypyrazoline, polypyrene, polypyridazine, polypyridine, polytriarylamine, poly(phenylene vinylene), poly(3-substituted thiophene), poly(3,4-bisubstituted thiophene), polyselenophene, poly(3-substituted selenophene), poly(3,4- bisubstituted selenophene), poly(bisthiophene), poly(terthiophene), poly(bisselenophene), poly(terselenophene), polythieno[2,3-b]thiophene, polythieno[3,2-b]thiophene, polybenzothiophene, polybenzo[1,2-b:4,5-b′]dithiophene, polyisothianaphthene, poly(monosubstituted pyrrole), poly(3,4-bisubstituted pyrrole), poly-1,3,4-oxadiazoles, polyisothianaphthene, derivatives and co-polymers thereof may be mentioned.

Preferred examples of donor polymers are copolymers of polyfluorenes and polythiophenes, each of which may be substituted, and polymers comprising benzothiadiazole-based and thiophene-based repeating units, each of which may be substituted.

A particularly preferred donor polymer comprises a repeat unit of formula (X):

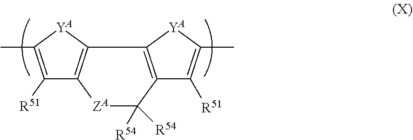

wherein Y^{A}, Z^{A}, R⁵¹ and R⁵⁴ are as described above.

Another particularly preferred donor polymer comprises repeat units of formula (XI):

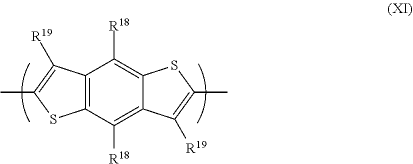

wherein R¹⁸ and R¹⁹ are each independently selected from H; F; C₁₋₁₂ alkyl wherein one or more non-adjacent, non-terminal C atoms may be replaced with O, S, COO or CO and one or more H atoms of the alkyl may be replaced with F; or an aromatic or heteroaromatic group Ar⁶ which is unsubstituted or substituted with one or more substituents selected from F and C₁₋₁₂ alkyl wherein one or more non-adjacent, non-terminal C atoms may be replaced with O, S, COO or CO.

The donor polymer is preferably a donor-acceptor (DA) copolymer comprising a donor repeat unit, for example a repeat unit of formula (X) or (XI), and an acceptor repeat unit, for example divalent electron-accepting units A¹ as described herein provided as polymeric repeat units.

### Organic Electronic Device

A compound of formula (I), (II), (I′) or (II′) may be provided as an active layer of an organic electronic device. In a preferred embodiment, a bulk heterojunction photoactive layer or an electron-accepting sub-layer of a photoactive layer of an organic photoresponsive device, more preferably an organic photodetector, comprises a compound of formula (I), (II), (I′) or (II′) as described herein.

A bulk heterojunction layer comprises or consists of an electron-donating material and an electron-accepting compound of formula (I), (II), (I′) or (II′) as described herein.

In some embodiments, the bulk heterojunction layer contains two or more accepting materials and/or two or more electron-accepting materials.

In some embodiments, the weight of the electron-donating material(s) to the electron-accepting material(s) is from about 1:0.5 to about 1:2, preferably about 1:1.1 to about 1:2.

Preferably, the electron-donating material has a type II interface with the electron-accepting material, i.e. the electron-donating material has a shallower HOMO and LUMO that the corresponding HOMO and LUMO levels of the electron-accepting material. Preferably, the compound of formula (I), (II), (I′) or (II′) has a HOMO level that is at least 0.05 eV deeper, optionally at least 0.10 eV deeper, than the HOMO of the electron-donating material.

Optionally, the gap between the HOMO level of the electron-donating material and the LUMO level of the electron-accepting compound of formula (I), (II), (I′) or (II′) is less than 1.4 eV.

Unless stated otherwise, HOMO and LUMO levels of materials as described herein are as measured by square wave voltammetry (SWV).

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

Ferrocene is added directly to the existing cell at the end of the experiment for calculation purposes where the potentials are determined for the oxidation and reduction of ferrocene versus Ag/AgCl using cyclic voltammetry (CV).

The sample is dissolved in toluene (3 mg/ml) and spun at 3000 rpm directly on to the glassy carbon working electrode.

LUMO=4.8-E ferrocene (peak to peak average)−E reduction of sample (peak maximum).

HOMO=4.8-E ferrocene (peak to peak average)+E oxidation of sample (peak maximum).

A typical SWV experiment runs at 15 Hz frequency; 25 mV amplitude and 0.004 V increment steps. Results are calculated from 3 freshly spun film samples for both the HOMO and LUMO data.

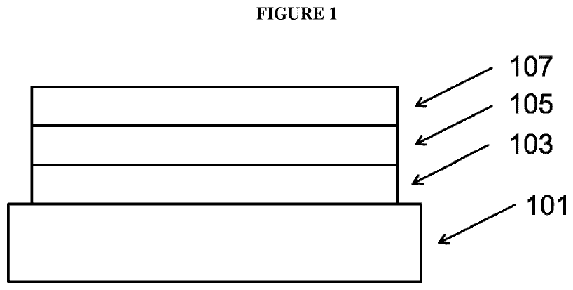

FIG. 1 illustrates an organic photoresponsive device according to some embodiments of the present disclosure. The organic photoresponsive device comprises a cathode 103, an anode 107 and a bulk heterojunction photoactive layer 105 disposed between the anode and the cathode. The organic photoresponsive device may be supported on a substrate 101, optionally a glass or plastic substrate. In other embodiments, the photoactive layer may comprise an electron-accepting sub-layer and an electron-donating sub-layer directly adjacent to and in contact with the electron-accepting sub-layer.

Each of the anode and cathode may independently be a single conductive layer or may comprise a plurality of layers.

At least one of the anode and cathode is transparent so that light incident on the device may reach the photoactive layer. In some embodiments, both of the anode and cathode are transparent. The transmittance of a transparent electrode may be selected according to an emission wavelength of a light source for use with the organic photodetector.

FIG. 1 illustrates an arrangement in which the cathode is disposed between the substrate and the anode. In other embodiments, the anode may be disposed between the cathode and the substrate.

The organic photoresponsive device may comprise layers other than the anode, cathode and photoactive layer shown in FIG. 1. In some embodiments, a hole-transporting layer and/or an electron-blocking layer is disposed between the anode and the photoactive layer. In some embodiments, an electron-transporting layer and/or a hole-blocking layer is disposed between the cathode and the photoactive layer. In some embodiments, a work function modification layer is disposed between the photoactive layer and the anode, and/or between the photoactive layer and the cathode.

The area of the OPD may be less than about 3 cm², less than about 2 cm², less than about 1 cm², less than about 0.75 cm², less than about 0.5 cm² or less than about 0.25 cm². Optionally, each OPD may be part of an OPD array wherein each OPD is a pixel of the array having an area as described herein, optionally an area of less than 1 mm², optionally in the range of 0.5 micron²-900 micron².

The substrate may be, without limitation, a glass or plastic substrate. The substrate can be an inorganic semiconductor. In some embodiments, the substrate may be silicon. For example, the substrate can be a wafer of silicon. The substrate is transparent if, in use, incident light is to be transmitted through the substrate and the electrode supported by the substrate.

The bulk heterojunction layer contains a polymer as described herein and an electron-accepting compound. The bulk heterojunction layer may consist of these materials or may comprise one or more further materials, for example one or more further electron-donating materials and/or one or more further electron-accepting compounds.

### Fullerene

In some embodiments, a compound of formula (I), (II), (I′) or (II′) is the only electron-accepting material of a bulk heterojunction layer or electron-accepting sub-layer as described herein.

In some embodiments, a bulk heterojuction layer or electron-accepting sub-layer contains a compound of formula (I), (II), (I′) or (II′) and one or more further electron-accepting materials. Preferred further electron-accepting materials are fullerenes. The compound of formula (I), (II), (I′) or (II′): fullerene acceptor weight ratio may be in the range of about 1:0.1-1:1, preferably in the range of about 1:0.1-1:0.5.

Fullerenes may be selected from, without limitation, C₆₀, C₇₀, C₇₆, C₇₈ and C₈₄ fullerenes or a derivative thereof, including, without limitation, PCBM-type fullerene derivatives including phenyl-C₆₁-butyric acid methyl ester (C₆₀PCBM), TCBM-type fullerene derivatives (e.g. tolyl-C₆₁-butyric acid methyl ester (C₆₀TCBM)), and ThCBM-type fullerene derivatives (e.g. thienyl-C₆₁-butyric acid methyl ester (C₆₀ThCBM).

Fullerene derivatives may have formula (V):

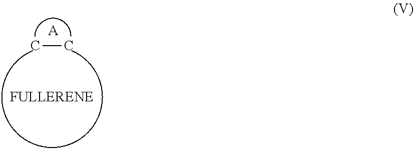

wherein A, together with the C-C group of the fullerene, forms a monocyclic or fused ring group which may be unsubstituted or substituted with one or more substituents.

Exemplary fullerene derivatives include formulae (Va), (Vb) and (Vc):

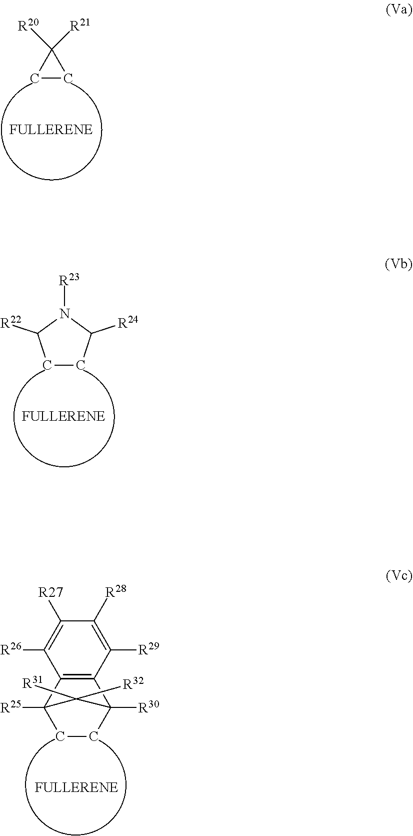

wherein R²⁰-R³² are each independently H or a substituent.

Substituents R²⁰-R³² are optionally and independently in each occurrence selected from the group consisting of aryl or heteroaryl, optionally phenyl, which may be unsubstituted or substituted with one or more substituents; and C₁₋₂₀ alkyl wherein one or more non-adjacent C atoms may be replaced with O, S, NR⁶, CO or COO and one or more H atoms may be replaced with F.

Substituents of aryl or heteroaryl, where present, are optionally selected from C₁₋₁₂ alkyl wherein one or more non-adjacent C atoms may be replaced with O, S, NR⁶, CO or COO and one or more H atoms may be replaced with F.

### Formulations

Formation of an electron-accepting sub-layer of a photoactive layer or a photoactive bulk heterojunction layer as described herein preferably comprises deposition of a formulation comprising electron-accepting material(s) including the compound of formula (I),(II), (I′) or (II′) and any other components of the electron-accepting sub-layer or bulk heterojunction layer dissolved or dispersed in a solvent or a mixture of two or more solvents. It will be understood that in the case of a bulk heterojunction layer the formulation further comprises one or more electron-donating materials.

The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

Preferably, the compound of formula (I), (II), (I′) or (II′) is deposited from a solution comprising at least one chlorinated and/or fluorinated solvent, for example a C₆₋₂₀ linear, branched or cyclic alkane or ether or benzene substituted with one or more F atoms and/or one or more Cl atoms. Preferred fluorinated solvents are perfluorinated solvents. Exemplary perfluorinated solvents include, without limitation, perfluorobenzene and perfluorinated C₆₋₂₀ linear, branched or cyclic alkanes.

The formulation may comprise a mixture of two or more solvents. In some embodiments, each solvent is halogenated, preferably fluorinated. In some embodiments, the formulation comprises one halogenated, preferably fluorinated, solvent and one non-halogenated, preferably non-fluorinated, solvent.

Exemplary non-halogenated solvents include, without limitation, benzene or naphthalene substituted with one or more substituents selected from chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives; and esters, optionally alkyl or aryl esters of alkyl or aryl carboxylic acids, optionally a C₁₋₁₀ alkyl benzoate.

The formulation may comprise further components in addition to the electron-accepting material, the one or more solvents and, in the case of a formulation of forming a bulk heterojunction layer, an electron-donating material. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

In some embodiments, formation of an organic photoactive device comprises, in any order, deposition of a first formulation comprising the compound of formula (I), (II), (I′) or (II′) and a halogenated, preferably fluorinated, solvent and evaporation of the or each solvent of the first formulation; and deposition of a second formulation comprising a non-fluorinated active organic material, for example an electron-donating material for forming an electron-donating sub-layer; or a charge-transporting or charge-blocking material for forming a charge-transporting or charge-blocking layer, dissolved in one or more non-halogenated, preferably non-fluorinated, solvents and evaporation of the or each solvent of the second formulation.

The layer or sub-layer containing the fluorinated compound of formula (I), (II), (I′) or (II′) is suitably not dissolved by the one or more non-halogenated solvents in the case where the second formulation is deposited onto this layer.

The layer or sub-layer containing the non-fluorinated active organic material is suitably not dissolved by the halogenated solvent of the first formulation in the case where the first formulation is deposited onto this layer.

### Applications

A circuit may comprise the OPD connected to one or more of a voltage source for applying a reverse bias to the device; a device configured to measure photocurrent; and an amplifier configured to amplify an output signal of the OPD. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source. In some embodiments, the light source has a peak wavelength of at least 900 nm or at least 1000 nm, optionally in the range of 900-1500 nm.

In some embodiments, the light from the light source may or may not be changed before reaching the OPD. For example, the light may be reflected, filtered, down-converted or up-converted before it reaches the OPD.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g., due to absorption by, reflection by and/or emission of light from an object, e.g. a target material in a sample disposed in a light path between the light source and the organic photodetector. The sample may be a non-biological sample, e.g. a water sample, or a biological sample taken from a human or animal subject. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor. The photodetector may be configured to detect light emitted from a target analyte which emits light upon irradiation by the light source or which is bound to a luminescent tag which emits light upon irradiation by the light source. The photodetector may be configured to detect a wavelength of light emitted by the target analyte or a luminescent tag bound thereto.

### EXAMPLES

### Compound Example 1

Compound Example 1 may be prepared according to the following reaction scheme:

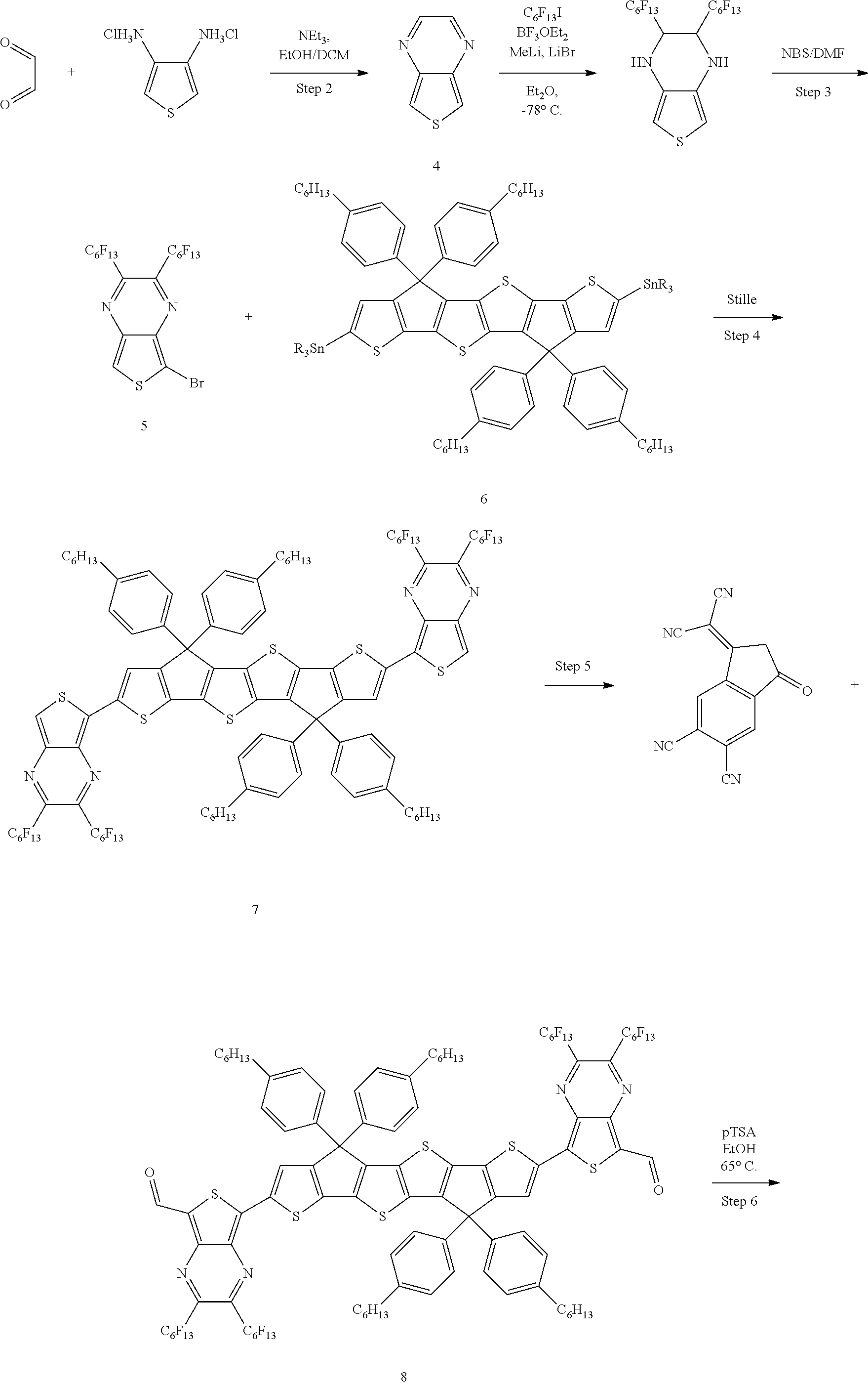

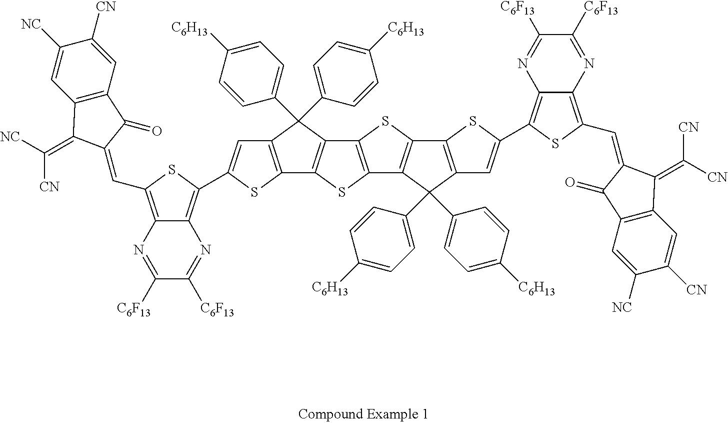

### Modelling Data

Energy levels of Model Example Compounds 1-4 and Model Comparative Compounds 1-7 were modelled using Gaussian09 software available from Gaussian using Gaussian09 with B3LYP (functional). Results are set out in Table 1 in which in which S1f corresponds to oscillator strength of the transition from S1 (predicting absorption intensity) and Eopt is the modelled optical gap.

TABLE 1

| | | | Eg | | Eopt |
|---|---|---|---|---|---|
| Structure | HOMO | LUMO | (nm) | S1 f | (nm) |
| 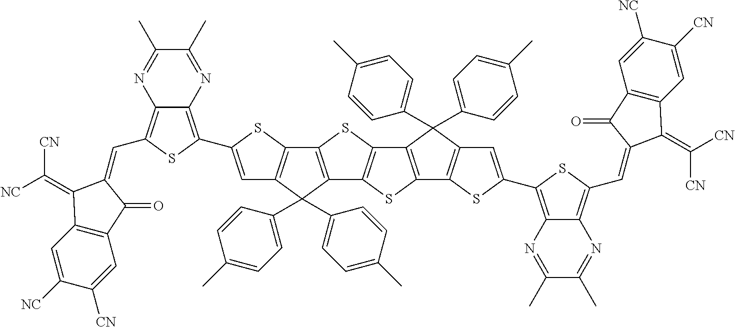 [SMILES: Cc1ccc(C2(c3ccc(C)cc3)c3cc(-c4sc(/C=C5\C(=O)c6cc(C#N)c(C#N)cc6C5=C(C#N)C#N)c5nc(C)c(C)nc45)sc3-c3sc4c5c(sc4c32)-c2sc(-c3sc(/C=C4\C(=O)c6cc(C#N)c(C#N)cc6C4=C(C#N)C#N)c4nc(C)c(C)nc34)cc2C5(c2ccc(C)cc2)c2ccc(C)cc2)cc1] | −5.40 | −3.97 | 865 | 3.29 | 927 |
| Comparative Model 1 | | | | | |
| 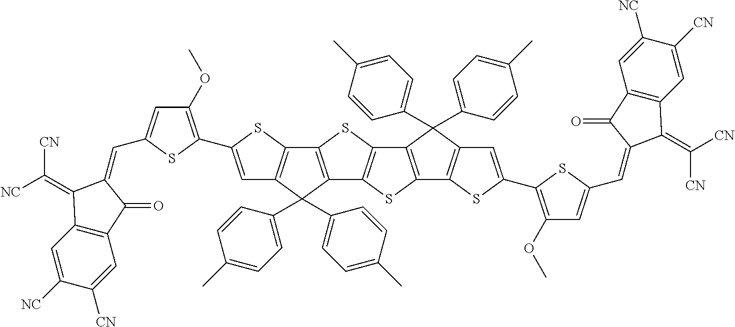 [SMILES: COc1cc(/C=C2\C(=O)c3cc(C#N)c(C#N)cc3C2=C(C#N)C#N)sc1-c1cc2c(s1)-c1sc3c4c(sc3c1C2(c1ccc(C)cc1)c1ccc(C)cc1)-c1sc(-c2sc(/C=C3\C(=O)c5cc(C#N)c(C#N)cc5C3=C(C#N)C#N)cc2OC)cc1C4(c1ccc(C)cc1)c1ccc(C)cc1] | −5.28 | −3.84 | 864 | 2.95 | 923 |
| Comparative Model 2 | | | | | |
| 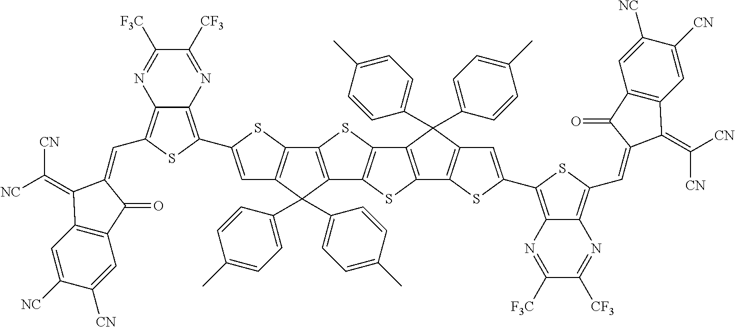 [SMILES: Cc1ccc(C2(c3ccc(C)cc3)c3cc(-c4sc(/C=C5\C(=O)c6cc(C#N)c(C#N)cc6C5=C(C#N)C#N)c5nc(C(F)(F)F)c(C(F)(F)F)nc45)sc3-c3sc4c5c(sc4c32)-c2sc(-c3sc(/C=C4\C(=O)c6cc(C#N)c(C#N)cc6C4=C(C#N)C#N)c4nc(C(F)(F)F)c(C(F)(F)F)nc34)cc2C5(c2ccc(C)cc2)c2ccc(C)cc2)cc1] | −5.75 | −4.41 | 923 | 3.05 | 979 |
| Model Compound 1 | | | | | |
| 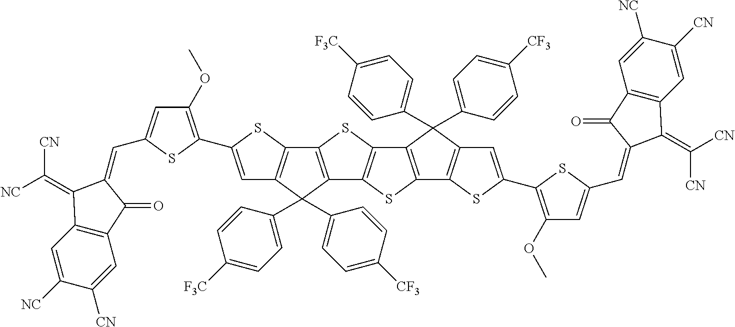 [SMILES: COc1cc(/C=C2\C(=O)c3cc(C#N)c(C#N)cc3C2=C(C#N)C#N)sc1-c1cc2c(s1)-c1sc3c4c(sc3c1C2(c1ccc(C(F)(F)F)cc1)c1ccc(C(F)(F)F)cc1)-c1sc(-c2sc(/C=C3\C(=O)c5cc(C#N)c(C#N)cc5C3=C(C#N)C#N)cc2OC)cc1C4(c1ccc(C(F)(F)F)cc1)c1ccc(C(F)(F)F)cc1] | −5.58 | −4.05 | 808 | 2.97 | 877 |
| Model Compound 2 | | | | | |
| 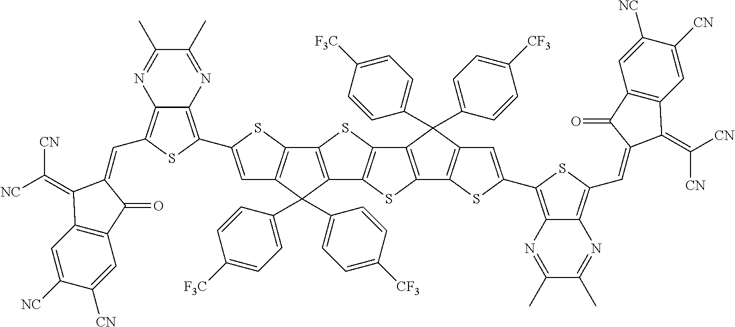 [SMILES: Cc1nc2c(/C=C3\C(=O)c4cc(C#N)c(C#N)cc4C3=C(C#N)C#N)sc(-c3cc4c(s3)-c3sc5c6c(sc5c3C4(c3ccc(C(F)(F)F)cc3)c3ccc(C(F)(F)F)cc3)-c3sc(-c4sc(/C=C5\C(=O)c7cc(C#N)c(C#N)cc7C5=C(C#N)C#N)c5nc(C)c(C)nc45)cc3C6(c3ccc(C(F)(F)F)cc3)c3ccc(C(F)(F)F)cc3)c2nc1C] | −5.69 | −4.17 | 820 | 3.22 | 892 |
| Model Compound 3 | | | | | |
| 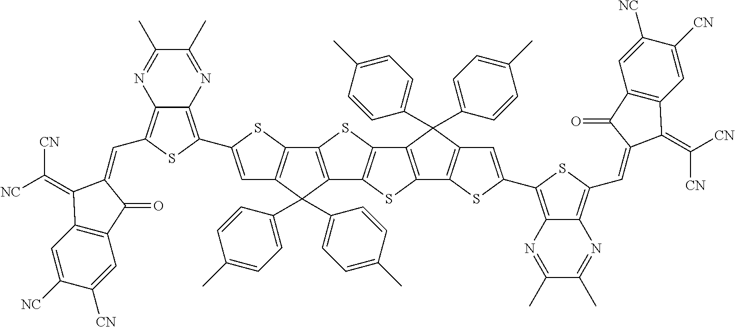 [SMILES: Cc1ccc(C2(c3ccc(C)cc3)c3cc(-c4sc(/C=C5\C(=O)c6cc(C#N)c(C#N)cc6C5=C(C#N)C#N)c5nc(C)c(C)nc45)sc3-c3sc4c5c(sc4c32)-c2sc(-c3sc(/C=C4\C(=O)c6cc(C#N)c(C#N)cc6C4=C(C#N)C#N)c4nc(C)c(C)nc34)cc2C5(c2ccc(C)cc2)c2ccc(C)cc2)cc1] | −5.40 | −3.97 | 863 | 3.29 | 926 |
| Comparative Model 3 | | | | | |
| 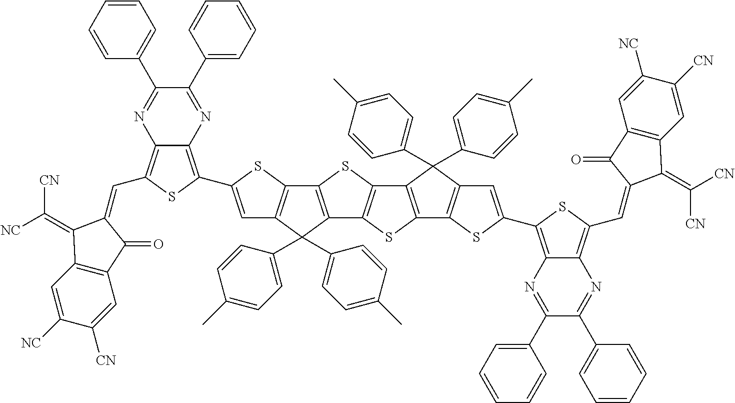 [SMILES: Cc1ccc(C2(c3ccc(C)cc3)c3cc(-c4sc(/C=C5\C(=O)c6cc(C#N)c(C#N)cc6C5=C(C#N)C#N)c5nc(-c6ccccc6)c(-c6ccccc6)nc45)sc3-c3sc4c5c(sc4c32)-c2sc(-c3sc(/C=C4\C(=O)c6cc(C#N)c(C#N)cc6C4=C(C#N)C#N)c4nc(-c6ccccc6)c(-c6ccccc6)nc34)cc2C5(c2ccc(C)cc2)c2ccc(C)cc2)cc1] | −5.39 | −3.98 | 883 | 3.13 | 958 |
| Comparative Model 4 | | | | | |

1. A compound of formula (I) or (II):
A¹-(B¹)x¹-(D¹)y¹-(B¹)×²-A¹ (I)
A¹-(B²)x⁵-(D²)y²-(B³)x³-A²-(B³)×⁴-(D³)y³-(B²)×⁶-A¹ (II)
wherein:
A¹ in each occurrence is independently a monovalent electron-accepting group;
A² is a divalent electron-accepting group;
D¹, D² and D³ independently in each occurrence is an electron-donating group;
y¹, y² and y³ are each independently at least 1;
B¹, B², and B³ independently in each occurrence is a bridging group;
x¹-x⁶ are each independently 0, 1, 2 or 3 with the provisos that:
in the case of the compound of formula (I) at least one of x¹ and x² is at least 1 and at least one B¹ is substituted with a fluorinated group; and
in the case of the compound of formula (II) at least one of x³, x⁴, x⁵ and x⁶ is at least 1 and at least one occurrence of at least one of B² and B³ is substituted with a fluorinated group.

2. The compound according to claim 1 wherein B¹, B² and B³ are each independently an optionally fused thiophene or optionally fused furan.

3. The compound according to claim 1 wherein the fluorinated group is selected from:
at least partially fluorinated C₁₋₂₀ alkyl in which one or more non-adjacent C atoms of a C₂₋₂₀ alkyl may be replaced with O, S, NR⁶, Si(R⁴)₂, CO, COO or CONR⁶ wherein R⁶ is H or a substituent and each R⁴ is independently a substituent, and
phenyl which is substituted with at least one of F and at least partially fluorinated C₁₋₂₀ alkyl in which one or more non-adjacent C atoms of a C₂₋₂₀ alkyl may be replaced with O, S, NR⁶, Si(R⁴)₂, CO, COO or CONR⁶.

4. The compound according to claim 1 wherein the fluorinated group is a perfluorinated group.

5. The compound according to claim 1 wherein the compound is a compound of formula (I) and wherein D1 is a group of formula (VIIe): 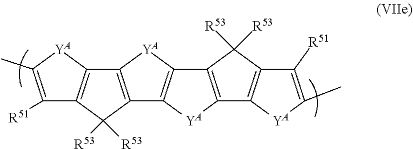
wherein Y^{A} is S or O, R⁵¹ is H or a substituent and R⁵³ is a substituent.

6. The compound according to claim 1 wherein A¹ is a group of formula (IXa-1): 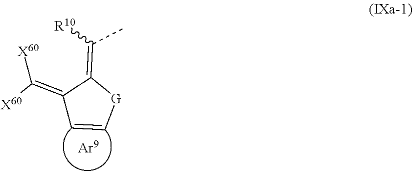
wherein:
G is C═O, C═S SO, SO₂, NR³³ or C(R³³)₂ wherein R³³ is CN or COOR⁴⁰ and R⁴⁰ is H or a substituent;
R¹⁰ is H or a substituent;
Ar⁹ is an unsubstituted or substituted monocyclic or fused aromatic or heteroaromatic group; and
X⁶⁰ are each independently CN, CF₃ or COOR⁴⁰.

7. A composition comprising an electron-donating material and an electron-accepting material wherein the electron-accepting material is a compound according to claim 1.

8. An organic electronic device comprising an active layer comprising a compound according to claim 1.

9. An organic electronic device comprising an active layer comprising a compound according to claim 1 wherein the organic electronic device is an organic photoresponsive device comprising a bulk heterojunction layer disposed between an anode and a cathode and wherein the bulk heterojunction layer comprises an electron-donating material and an electron-accepting material wherein the electron-accepting material is a compound according to claim 1.

10. An organic electronic device according to claim 9 wherein the organic photoresponsive device is an organic photodetector.

11. A formulation comprising a compound according to claim 1 dissolved or dispersed in one or more solvents.

12. The formulation according to claim 11 wherein the one or more solvents include at least one halogenated solvent.

13. The formulation according to claim 12 wherein the halogenated solvent is a fluorinated solvent.

14. A method of forming an organic electronic device comprising an active layer comprising a compound according to claim 1 wherein formation of the active layer comprises deposition of a formulation comprising the compound according to claim 1 onto a surface and evaporation of the one or more solvents.

15. The method according to claim 14 wherein the formulation is deposited onto an organic layer comprising a non-fluorinated active material.

16. The method according to claim 14 wherein, following formation of the active layer, a further formulation comprising a non-fluorinated active material dissolved in one or more non-fluorinated solvents is deposited onto the active layer.

17. An organic photoresponsive device comprising an anode, a cathode and a photoactive layer disposed between the anode wherein the photoactive layer comprises an electron-donating sub-layer comprising an electron-donating compound and an electron-accepting sub-layer comprising a compound of formula (I′) or (II′) directly adjacent to and in contact with the electron-donating sub-layer:
A¹-(B¹)x¹¹-(D¹)y¹-(B¹)x¹²-A¹ (I′)
A¹-(B²)x¹⁵-(D²)y²-(B³)x¹³-A²-(B³)x¹⁴-(D³)y³-(B²)x¹⁶-A¹ (II′)
wherein:
A¹ in each occurrence is independently a monovalent electron-accepting group;
A² is a divalent electron-accepting group;
D¹, D² and D³ independently in each occurrence is an electron-donating group;
y¹, y² and y³ are each independently at least 1;
B¹, B², and B³ independently in each occurrence is a bridging group;
x¹¹-x¹⁶ are each independently 0, 1, 2 or 3; and
the compound of formula (I′) or (II′) is substituted with at least one fluorinated group.

18. The organic photoresponsive device according to claim 17 wherein the electron-donating compound is not substituted with a fluorinated group.

19. (canceled)

20. A method of forming the organic photoresponsive device according to claim 17 wherein formation of the electron-accepting sub-layer comprises deposition of a formulation comprising a halogenated solvent and the compound of formula (I′) or (II′) dissolved in the formulation, and wherein formation of the electron-donating sub-layer comprises deposition of a formulation comprising one or more solvents selected from non-halogenated solvents and the electron-donating compound.

21. A photosensor comprising a light source and an organic photodetector according to claim 10 wherein the organic photodetector is configured to detect light emitted from the light source and wherein the light source emits light having a peak wavelength of greater than 900 nm.
